(19) United States
(12) Patent Application Publication
(10) Pub. No.: US 2011/0035155 A1
(43) Pub. Date: Feb. 10, 2011

(54) PROTEIN AGGREGATION PREDICTION SYSTEMS

(75) Inventors: Michele Vendruscolo, Cambridgeshire (GB); Christopher Dobson, Cambridgeshire (GB); Gian Gaetano Tartaglia, Barcelona (ES); Sebastian Pechmann, Cambridgeshire (GB)

Correspondence Address: SCHWEGMAN, LUNDBERG & WOESSNER, P.A., P.O. BOX 2938, MINNEAPOLIS, MN, 55402, US

(73) Assignee: Cambridge Enterprise Limited, Cambridge (GB)

(21) Appl. No.: 12/745,186

(22) PCT Filed: Nov. 13, 2008
(86) PCT No.: PCT/GB08/51055

§ 371 Date: Oct. 26, 2010

(30) Foreign Application Priority Data
Nov. 28, 2007 (GB) 0723288.7

(51) Int. Cl. G06F 19/00; G01N 33/50

(52) U.S. Cl. 702/19

(57) ABSTRACT

We describe methods for identifying aggregation-prone regions in structured—that is folded—proteins. Embodiments of the method use a local propensity for aggregation (Aᵢ) at an amino acid position, this being determined by a combination of a hydrophobicity value, an α-helix propensity value, a β-sheet propensity value, a charge value and a pattern value for the amino acid position. This is combined with local structural stability values for the amino acid positions to identify one or more regions in the amino acid sequence which, in the folded protein, are predicted to promote aggregation.

### FIELD OF THE INVENTION

This invention relates to methods for identifying aggregation-prone regions in structured (folded) proteins and to related methods for determining the aggregation propensity of a protein, to computer program code and equipment for implementing the methods, and to related methods of identifying new drugs and drug targets as well as protein toxicities.

### BACKGROUND TO THE INVENTION

Background prior art is described in Protein Science, Vol 15, 2006, J A Marsh et al, “Sensitivity of secondary structure propensities to sequence differences between alpha- and gamma-synuclein: Implicationd for fibrillation”, 2795-2804; and in silico Biology, Vol 7, 2007, S Inicula-Thomas et al, “Correlation between the structural stability and aggregation propensity of proteins”, 225-237. We have previously described, in WO 2004/066168 and in WO 2005/045442, techniques for predicting the rate of aggregation/solubility of proteins in their native, unfolded state. These techniques are useful, for example, in predicting aggregation-resistant mutational variants of unstructured polypeptide chains but they are not in general applicable to the prediction of aggregation in structured (folded) proteins. However, the aggregation of proteins from their folded state is important for many diseases, and the accurate prediction of this phenomenon is considered a difficult problem which, heretofore, has not been solved. We will describe a tool to address this problem; there are many applications of the tool, including rational design of drugs as well as protein production techniques.

### SUMMARY OF THE INVENTION

According to a first aspect of the invention there is therefore provided a method of identifying one or more regions in the amino acid sequence of a protein which, in the folded protein, are predicted to promote aggregation, the method comprising: determining, for amino acid positions (i) along said sequence, a local propensity for aggregation (Aᵢ) at a said amino acid position, said local propensity for aggregation being determined by a combination of a hydrophobicity value, an α-helix propensity value, a β-sheet propensity value, a charge value and a pattern value for said amino acid position; determining local structural stability values for said amino acid positions, a said local structural stability value comprising a measure of local structural stability at a said amino position; and combining said determined local propensities for aggregation at said amino acid positions and said local structural stability values at said amino acid positions to identify one or more regions in said amino acid sequence which, in said folded protein, are predicted to promote aggregation.

The local structural stability values take into account that the protein is in its folded state. In some preferred embodiments this information is predicted purely from the amino acid sequence of the protein. In preferred embodiments a local structural stability value effectively measures the amplitude of thermal fluctuations of the structure. In some particularly preferred embodiments a local structural stability value at a position i in the sequence (Pᵢ) is a property of the amino acid sequence (in general substantially the entire amino acid sequence). A value for logarithm Pᵢ, which is determined by the propensity of the protein to fold and remain stable in its folded state, is determined by the CamP method as described in Tartaglia, G. G., Cavalli, A. & Vendruscolo, M. (2007) Structure 15, 139-143, the contents of which are hereby incorporated by reference. In embodiments of the method the local structural stability values are determined without knowledge of the native folded structure of the protein.

The combination of the determined local propensities for aggregation and the local structural stability values is, in embodiments, performed by modulating the local propensities for aggregation with the local structural stability values although, potentially, the combination may be made in other ways for example by representing the two sets of values on different axes of a graphical representation of the data. The skilled person will appreciate that the determination of a local propensity for aggregation need not comprise a linear combination of the hydrophobicity, α-helix and β-sheet propensity, charge and pattern values. In some preferred embodiments the local propensities for aggregation modulated by the local structural stability data is used to determine an aggregation propensity profile for the folded protein representing variations in the combined data with position along the sequence. One or more regions which are predicted to be prone to aggregation may then be readily identified by identifying local or absolute maxima, for example local peaks in the profile or regions of the profile where the profile has a value greater than a threshold level.

Preferred embodiments of the method also take into account the concept of gatekeepers, in particular by taking into account the effect of local charge on the effect of amino acid patterns. Thus whilst some amino acid patterns, for example a pattern alternating hydrophilic and hydrophobic amino acids, in particular with a length of at least 5 amino acids, promote aggregation this effect is suppressed by local charge either flanking or inside the pattern. Thus preferred embodiments of the method determine a total local charge within a window to either side of the amino acid pattern and use this value to modify the determined local propensities for aggregation at an amino acid position.

Thus in a further aspect the invention provides a method of identifying one or more regions in the amino acid sequence of a protein which, in the folded protein, are predicted to promote aggregation, the method comprising: determining, for a plurality of positions, i, along said sequence, a value of pᵢ^{agg}, where pᵢ^{agg} represents an intrinsic aggregation propensity of an amino acid at position i and comprises a function of pₕ, pₛ, p_{hyd} and p_{c} and pₕ, pₛ, p_{hyd} and p_{c} are, respectively, an α-helix propensity value, a β-sheet propensity value, a hydrophobicity value, and a charge value for an amino acid at a said position i along said sequence; determining, for a plurality of positions, i, along said sequence, a value of Aᵢ^{p}, where Aᵢ^{p} is determined from

$${α}_{1} {\sum }_{window 1} {p}_{i}^{agg} + {α}_{pat} {I}_{i}^{pat} + {α}_{gk} {I}_{i}^{gk}$$

where

$${\sum }_{window 1}$$

denotes a first sum over amino acid positions in a first window to either side of position i, Iᵢ^{pat} is a pattern value representing a pattern of one or both of hydrophilic and hydrophobic amino acids at position i, Iᵢ^{gk} is a charge value representing a charge flanking or inside a said pattern, and wherein α₁, αₚₐₜ and α_{gk} are scaling factors; and determining an aggregation propensity profile for said protein from values of Aᵢ^{p} for said plurality of positions i along said sequence, said aggregation propensity profile comprising data identifying a variation of relative aggregation propensity with position along said sequence.

As previously mentioned, the skilled person will appreciate that a wide range of functions of pₕ, pₛ, p_{hyd} and p_{c} may be employed and embodiments of the technique are not limited to a linear combination of these values. This embodiments of the method are not restricted to the specific form of the calculation of Pᵢ^{agg} given in Equation (1) later.

As mentioned above, preferably the charge value representing a charge flanking or inside a local pattern of amino acids comprises a sum of (amino acid) charges over a window at an amino acid position i; preferably this (second) window is larger than the (first) window used to determine Aᵢ^{p}. In embodiments the first window has a length substantially equal to the persistence length of a β strand, for example seven amino acids; in embodiments an edge of a second window is the point at which the “memory” effect of the charge on the β strand is effectively lost, for example at more than three, five or seven amino acids beyond a boundary of the first window.

In preferred embodiments the determining of the aggregation propensity profile takes into account structural protection and aggregation propensity at a residue-specific level, in particular by multiplying by

$$( {α}_{2} - \frac{logarithm {P}_{i}}{{α}_{3}} )$$

Here α₂ and α₃ are scaling factors and the logarithm may, for example, be either to base 10 or to base e (the logarithm effectively takes account of measuring populations/probability and transferring to a free energy representation which represents stability); in embodiments the protection factor Pᵢ represents protection from hydrogen exchange and the free energy relates to the free energy contribution of creating a vanderWaals contact or hydrogen bond. The larger the logarithm Pᵢ term the more unstable the native structure; in embodiments α₃ has a value of approximately 15 since, experimentally, it has been found that values of logarithm Pᵢ larger than this correspond to unstable local structure. In embodiments of the method a normalized intrinsic aggregation propensity profile Zᵢ^{p} may be determined, but the skilled person will appreciate that normalization is not essential. Likewise is not necessary to explicitly determine such a normalized intrinsic aggregation propensity profile prior to modulating by local structural stability values.

In embodiments of the above-described techniques an overall aggregation propensity may be determined by summing aggregation propensity data, preferably taking into account the local structural stability values, performing the summing over only those regions identified as predicted to promote aggregation.

Thus in a further aspect the invention provides a method of determining the overall aggregation propensity of a folded protein, the method comprising: identifying one or more regions in the amino acid sequence of a protein which, in the folded protein, are predicted to promote aggregation taking into account one or both of a local hydrogen exchange and the suppression of an aggregation-inducing amino acid pattern by local charge; and then summing aggregation propensity data determined from values of a local propensity for aggregation (Aᵢ) at a plurality of amino acid positions (i) along said sequence; wherein said summing comprises summing over substantially only said identified regions.

The determined overall aggregation propensity of a protein, predicted from its amino acid sequence, may be used to identify a polypeptide sequence which is particularly suitable (or unsuitable) for manufacture because it is unlikely (or likely) to form insoluble aggregates. Having identified polypeptides suitable for manufacture embodiments of the method may then be employed to make a polypeptide (protein) identified in this way. In some preferred techniques such an identified polypeptide is manufactured using robotic polypeptide synthesis apparatus, for example under the control of computer program code to implement a method as described above. Additionally automatic (robotic) laboratory equipment may be controlled by computer program code configured to implement a method as described above to identify one or more regions in the amino acid sequence of a protein which, in the folded protein, are predicted to promote aggregation. Such equipment may be employed, for example, automatically to identify a drug target in a protein and/or automatically to identify a drug which interacts with the protein, in particular at one or more identified target regions.

Thus in another aspect the invention provides a method of identifying a drug target in a protein, in particular using a method as described above to identify one or more target portions of the amino acid sequence which are predicted to promote aggregation. Having made such a prediction, optionally this can be tested by, for example, mutating the sequence. Still further, having identified one or more drug targets in the protein, the method may then continue to identify one or more drugs predicted to interact with the protein, for example by binding at the target site. This may be as straightforward as looking in a database to determine whether there are any molecules which are known to bind at the target site, or a rational approach to identifying a molecule to bind at the target may be employed once the target site has been identified, or an in-vivo/in-vitro screening approach may be employed. Again, such a procedure may be implemented by automatic (robotic) laboratory equipment, for example under the control of computer program code to implement a method as described above.

Thus the invention further provides computer program code for controlling a computer or computerized apparatus to implement a method or system as described above. The code may be provided on a carrier such as a disk, for example a CD- or DVD-ROM, or in programmed memory for example as Firmware. Code (and/or data) to implement embodiments of the invention may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog (Trade Mark) or VHDL (Very high speed integrated circuit Hardware Description Language). As the skilled person will appreciate such code and/or data may be distributed between a plurality of coupled components in communication with one another.

The skilled person will understand that features of the above-described aspects and embodiments of the invention may be combined in any permutation.

### BRIEF DESCRIPTION OF THE DRAWINGS

These and other aspects of the invention will now be further described, by way of example only, with reference to the accompanying figures in which:

FIGS. 1a and 1b show, respectively, a block diagram of a computer system for implementing an embodiment of a method according to the invention; and aggregation propensity profiles of four peptides involved in amyloid diseases: the upper lines indicate the intrinsic aggregation propensity profiles, Z^{p} and the lower lines the aggregation propensities, Z^{ps}, the latter calculated by taking into account the structural protection provided by the globular structure of the folded form of the protein; Aβ₁₋₄₂: shaded regions indicate the segments that form the cross-β core and the bar indicates the region corresponding to the peptide Aβ₁₆₋₂₂ (KLVFFAE) which has been shown to form highly regular amyloid fibrils; glucagon; calcitonin; the second WW domain of human CA150 where shaded regions indicate the segments that form the cross-β core;

FIG. 2 shows examples of the predicted aggregation propensity profiles of structured proteins: the regions of low folding propensities, which are less protected from aggregation, are identified as the highest peaks in the aggregation propensity profiles Z^{PS} (black lines) calculated by taking into account the structural protections in the folded form; the intrinsic aggregation propensity profiles Z^{P} are the upper lines; secondary structure elements are shown as bars200 (β sheets) and the upper bars 202 (α helices); Lysozyme: the shaded areas represent the regions of residues 26-123 and 32-108, which are important for aggregation; Myoglobin: the shaded area identifies a highly aggregation-prone peptide fragment (residues 100-114);

FIG. 3 shows relationships between folding (log P score) and aggregation (Z^{P} score) propensities at the individual residue level (H=helix, S=strand, and T=turn); unstructured regions according to www.expasy.org are marked with stars. (a) Lysozyme: we predict the regions or residues 43-54 (helix), 73-76 (turn), 82-85 (strand), and 96-98, (unstructured) to have simultaneously low structural protection and high aggregation propensity, and therefore be particularly prone to aggregate under destabilizing condition; we also label with a number the positions associated to amyloidogenic mutations; the residue numbering in the figure follows that on the ExPASy web server, and includes a 18-residue N-terminal tag. (b) Myoglobin: we predict the regions of residues 4-19 (helix), 21-35 (helix), 125-149 (helix) to have high aggregation propensity and low structural protection;

FIG. 4 shows aggregation propensity profiles of two prion proteins for which detailed structural information is available; the upper lines indicate the intrinsic aggregation propensity profiles, Z^{P}, and the lower lines the aggregation propensity profiles, Z^{PS}, calculated by taking into account the structural protection provided by the globular structure of the folded form of the protein. (a) Aggregation propensity profile for the sequence of hPrP₍₂₃₋₂₃₁₎; intrinsic profile Z^{P} and effective Z^{PS} profile; the secondary structure elements present in hPrPC are indicated as bars 400 (β-sheets) and bars 402 (α helices). The position of the disulfide bond C179-C214 is indicated by a line 404. The experimentally-determined sensitive region for aggregation (residues 113-127) is indicated by a gray shaded area, and it is shown to overlap substantially with the major region predicted by our method to have a significant aggregation propensity (Z^{PS}>1). (b) HET-s: The regions corresponding to the four β strands identified by solid-state NMR are indicated; the shaded region corresponds to the C-terminal fragment whose amyloid structure has been characterised through solid-state NMR spectroscopy.

FIG. 5 shows a relationship between folding (log P score) and aggregation (Z^{P} score) propensities at the individual residue level for the human prion protein (H=helix, S=strand, and T=turn); unstructured regions according to www.expasy.org are marked with stars; We predict that the region of residues 120-123 has the highest aggregation propensity and the lowest structural protection, followed by the region of repeat 84-91; we also label the positions associated to the CJD mutations.

### DETAILED DESCRIPTION OF PREFERRED EMBODIMENTS

We will describe a method of predicting the regions of the sequences of peptides and proteins that are most important in promoting their aggregation and amyloid formation. The method allows such predictions to be carried out for conditions under which the molecules concerned can contain a significant degree of persistent structure. In order to achieve this result embodiments of the method use only a knowledge of the sequence of amino acids to estimate simultaneously both the propensity for folding and for aggregation, as well as the way in which these two types of propensity compete. We illustrate the approach by its application to a set of peptides and proteins both associated and not associated with disease. The results show not only that the regions of a protein with a high intrinsic aggregation propensity can be identified in a robust manner, but also that the structural context of such regions in the monomeric (soluble) form is very important for determining their role in the aggregation process.

Specific regions of the amino acid sequences of polypeptide chains, sometimes known as “aggregation-prone” regions (Pawar, A. P., DuBay, K. F., Zurdo, J., Chiti, F., Vendruscolo, M. & Dobson, C. M. (2005) J. Mol. Biol. 350, 379-392), play a major role in determining their tendency to aggregate and ultimately to form organized structures such as amyloid fibrils (Pawar, A. P., DuBay, K. F., Zurdo, J., Chiti, F., Vendruscolo, M. & Dobson, C. M. (2005) J. Mol. Biol. 350, 379-392; de Groot, N. S., Pallares, I., Aviles, F. X., Vendrell, J. & Ventura, S. (2005) BMC Struct. Biol. 5; Fernandez-Escamilla, A. M., Rousseau, F., Schymkowitz, J. & Serrano, L. (2004) Nat Biotech 22, 1302-1306). Strong support for this view has been provided by analyzing the effects of mutations on the aggregation propensities of specific peptides and proteins (Chiti, F., Taddei, N., Baroni, F., Capanni, C., Stefani, M., Ramponi, G. & Dobson, C. M. (2002) Nat. Struct. Biol. 9, 137-143) and through the determination of high-resolution structural models that illustrate that specific segments of polypeptide chains constitute the highly ordered cores of the fibrils. The existence of aggregation-prone regions has suggested the way in which rational mutagenesis can reduce the problem of aggregation in biotechnology (Ventura, S. & Villaverde, A. (2006) Trends Biotech. 24, 179-185). In addition it has suggested therapeutic strategies that specifically target these regions in order to reduce their tendency to promote the formation of ordered intermolecular assemblies (Tatarek-Nossol, M., Yan, L. M., Schmauder, A., Tenidis, K., Westermark, G. & Kapurniotu, A. (2005) Chemistry & Biology 12, 797-809).

The main physico-chemical factors that promote the aggregation of unfolded polypeptide chains have recently been characterized (Chiti, F., Stefani, M., Taddei, N., Ramponi, G. & Dobson, C. M. (2003) Nature, 424, 805-808.

Dubay, K. F., Pawar, A. P., Chiti, F., Zurdo, J., Dobson, C. M. & Vendruscolo, M. (2004) J. Mol. Biol. 341, 1317-1326) and on this basis several algorithms have been proposed to predict “aggregation propensity profiles” that enable the identification of regions with a high intrinsic propensity for aggregation (Rousseau, F., Schymkowitz, J. & Serrano, L. (2006) Curr. Op. Struct. Biol. 16, 118-126; Tartaglia, G. G., Cavalli, A., Pellarin, R. & Caflisch, A. (2004) Protein Sci. 13, 1939-1941; Thompson, M. J., Sievers, S. A., Karanicolas, J., Ivanova, M. I., Baker, D. & Eisenberg, D. (2006) Proc. Natl. Acad. Sci. USA 103, 4074-4078; Trovato, A., Chiti, F., Maritan, A. & Seno, F. (2006) PLoS Comp. Biol. 2, 1608-1618; Conchillo-Sole, O., de Groot, N. S., Aviles, F. X., Vendrell, J., Daura, X. & Ventura, S. (2007) BMC Bioinformatics 8). We have previously shown the strength of this approach for predicting the aggregation-prone regions of polypeptide chains that are unstructured under physiological conditions, including the Aβ peptide associated with Alzheimer's disease and α-synuclein, a natively unfolded protein whose aggregation has been linked to Parkinson's disease.

We now extend this approach for predicting the regions that promote the aggregation of structured and partially structured globular proteins. In such an enterprise we consider the possibility that regions with a high intrinsic propensity for aggregation may be buried inside stable and often highly cooperative structural elements, and therefore be unable in such states to form the specific intermolecular interactions that lead to aggregation. Shielded in this way, therefore, they may become unable to play a major role in the aggregation process, although, following mutations that destabilize the native structure, they might acquire this ability. In order to be able to take into consideration the tendency of a given region of a protein sequence to adopt a folded conformation, we exploit the possibility of predicting the local stability of the various regions of a protein from the knowledge of its sequence (Tartaglia, G. G., Cavalli, A. & Vendruscolo, M. (2007) Structure 15, 139-143). In essence, given the amino acid sequence of a protein, we show here how it is possible to combine the predictions of the propensity profiles for forming ordered aggregates and for folding into stable structures. We illustrate this approach through its application to the prediction of aggregation profiles for a range of peptides and proteins whose aggregation propensities have been characterized experimentally in particular detail. Since the algorithm that we developed is based on mutational data relative to the kinetics of amyloid formation, the results that we present enable us to discuss how regions that have a high propensity to promote the aggregation process may be distinct from those that play a major role in stabilizing the structural cores of the amyloid conformations.

Procedure

Intrinsic Propensity Profiles for Aggregation of Polypeptide Sequences

In the approach described in this paper, the intrinsic aggregation propensities of individual amino acids are defined as (1)

pᵢ^{agg}=αₕpₕ+αₛpₛ+α_{hyd}p_{hyd}+α_{c}p_{c} (1)

where pₕ and pₛ are the propensities for α helix and β sheet formation, respectively and p_{hyd} is the hydrophobicity, and p_{c} is the charge. These propensities are then combined in a linear way with coefficients α determined as described below. The skilled person will understand that other than linear models may be employed. The pi^{agg} values are combined to provide a profile, Aₚ, which describes the intrinsic propensity for aggregation as a function of the complete amino acid sequence (1). In embodiments pi^{agg} may be scaled, for example to be between ±1, using coefficients α. At each position i along the sequence we define the profile Aₚ as an average over a window of seven residues

$${A}_{i}^{p} = \frac{1}{7} {\sum }_{j = - 3}^{3} {p}_{i + j}^{agg} + {α}_{pat} {I}_{i}^{pat} + {α}_{gk} {I}_{i}^{gk} \qquad ( 2 )$$

where Iₚₐₜ is the term that takes into account the presence of specific patterns of alternating hydrophobic and hydrophilic residues (1) and I_{gk} is the term that takes into account the gatekeeping effect of individual charges cⁱ

$${I}_{i}^{gk} = {\sum }_{j = - 10}^{10} {c}_{i + j} \qquad ( 3 )$$

The parameters α may be fitted according to the general procedure described by DuBay et al. (16. Dubay, K. F., Pawar, A. P., Chiti, F., Zurdo, J., Dobson, C. M. & Vendruscolo, M. (2004) J. Mol. Biol. 341, 1317-1326). In order to compare the intrinsic propensity profiles we normalize Ap by considering the average (μ_{A}) and the standard deviation (σ_{A}) of Aₖ^{p} at each position k for random sequences. We thus obtain the normalized intrinsic aggregation propensity profile

$${Z}_{i}^{p} = \frac{{A}_{i}^{p} - μ}{σ} \qquad ( 4 )$$

The aim is for Zᵢ^{p} to have an average of zero and a standard deviation of 1

where we calculated the average μ and the standard deviation σ over random sequences

$$μ = \frac{1}{( N - 8 ) \cdot {N}_{s}} {\sum }_{k = 1}^{{N}_{s}} {\sum }_{i = 4}^{N - 4} {A}_{i}^{p} ( {S}_{k} ) \\ {σ}^{2} = \frac{1}{( N - 8 ) \cdot {N}_{s}} {\sum }_{k = 1}^{{N}_{s}} {\sum }_{i = 4}^{N - 4} {( {A}_{i}^{p} ( {S}_{k} ) - μ )}^{2} \qquad ( 5 )$$

In these formulas, we considered Nₛ random sequences of length N, and we verified that μ and σ are essentially constant for values of N ranging from 50 to 1000. The values of μ and σ depend on the length N; for example for N=100, μ=6.9 and σ=7.3. Random sequences were generated by using the amino acid frequencies of the SWISS-PROT database (Boeckmann, B., Bairoch, A., Apweiler, R., Blatter, M. C., Estreicher, A., Gasteiger, E., Martin, M. J., Michoud, K., O'Donovan, C., Phan, I., Pilbout, S. & Schneider, M. (2003) Nucleic Acids Res. 31, 365-370).

Prediction of Folding Propensities from the Sequence

We used the CamP method, by which the flexibility and the solvent accessibility of proteins are predicted with high accuracy. This method enables the prediction from the knowledge of amino acid sequence of the buried regions with more than 80% accuracy and of the protection factors for hydrogen exchange with an average 60% accuracy (Tartaglia, G. G., Cavalli, A. & Vendruscolo, M. (2007) Structure 15, 139-143).

Prediction of Aggregation Propensity Profiles for Partially Structured Polypeptide Chains

A region of a polypeptide sequence should meet two conditions in order to promote aggregation: it should have a high intrinsic aggregation propensity (Z^{P}>0) and it should be sufficiently unstable to have a significant propensity to form intermolecular interactions. In order to describe the latter we use the CamP method for the protection factors from hydrogen exchange, ln P. For those values that have Z^{P}>0 we modified the aggregation propensity profile Z^{P} by modulating with ln P

$${Z}_{i}^{ps} = {Z}_{i}^{p} ( 1 - \frac{1 {nP}_{i}}{15} ) \qquad ( 6 )$$

Absolute Propensities for Aggregation of Structured Polypeptide Sequences

Only residues with low local stabilities are considered as contributing to the overall aggregation propensity, Z_{agg}^{s}, resulting in the formula

$${Z}_{agg}^{s} = \frac{{\sum }_{i = 1}^{N} {Z}_{i}^{ps} ϑ ( {Z}_{i}^{ps} )}{{\sum }_{i = 1}^{N} ϑ ( {Z}_{i}^{ps} )} \qquad ( 7 )$$

where the function θ(x) is 1 for x>0 and 0 for x<0. We use a similar expression (see “Systematic In Vivo Analysis of the Intrinsic Determinants of Amyloid β Pathogenicity” Leila M. Luheshi, Gian Gaetano Tartaglia, Ann-Christin Brorrsson, Amol P. Pawar, Ian E. Watson, Fabrizio Chiti, Michele Vendruscolo, David A. Lomas, Christopher M. Dobson, Damian C. Crowther, PloS Biology (www.plosbiology.org), November 2007, Volume 5, Issue 11, e290) for computing the absolute aggregation propensity without structural corrections

$${Z}_{agg} \frac{{\sum }_{i = 1}^{N} {Z}_{i}^{p} ϑ ( {Z}_{i}^{p} )}{{\sum }_{i = 1}^{N} ϑ ( {Z}_{i}^{p} )} \qquad ( 8 )$$

Example Computer System Implementing the Above-Described Method

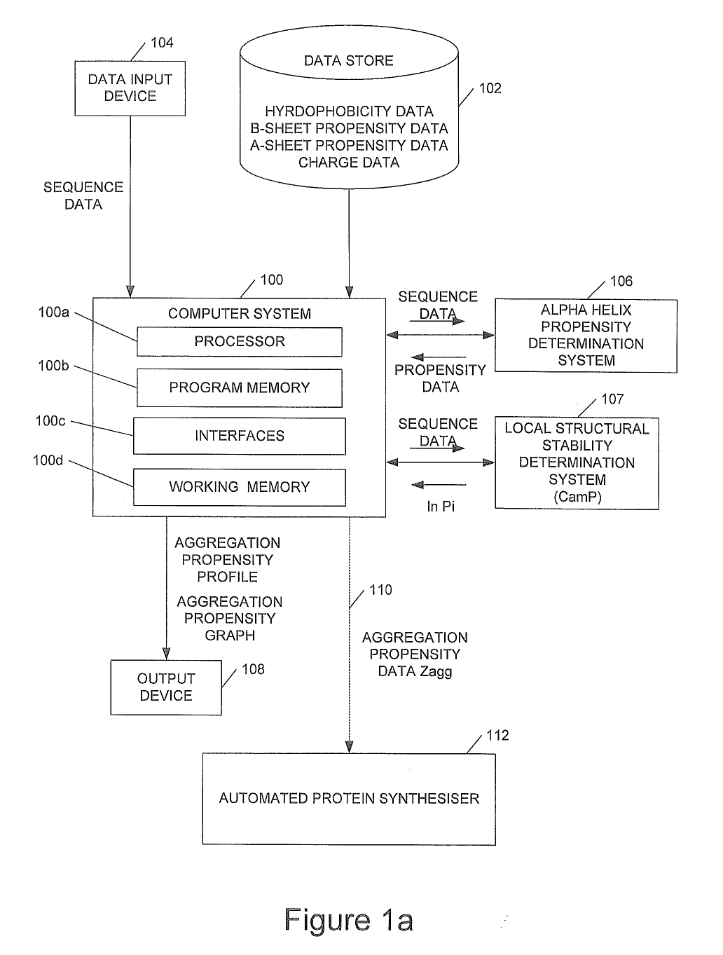

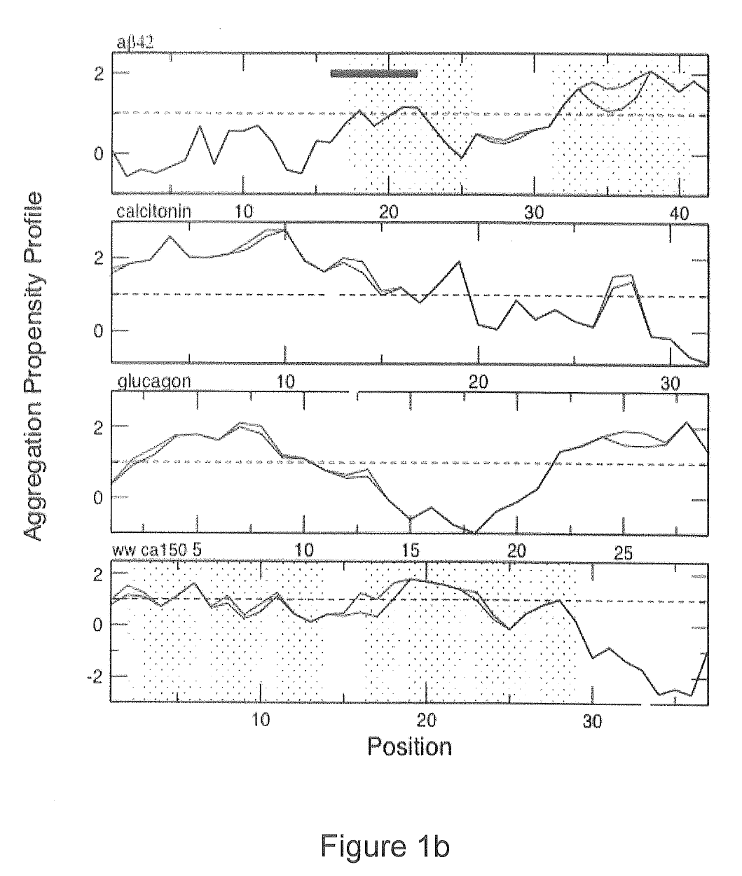

Referring now to FIG. 1a, this shows a block diagram of a computer system for implementing the above-described method. A general purpose computer system 100 comprises a processor 100a coupled to programme memory 100b storing computer programme code to implement the method, to working memory 100d, and to interfaces 100c such as conventional computer screen, keyboard, mouse, and printer, as well as other interfaces such as a network interface, and software interfaces such as a database interface.

The computer system 100 accepts user input from a data input device 104 such as a keyboard, input data file, or network interface, and provides an output to an output device 108 such as a printer, display, network interface, or data storage device. Input device 104, for example a network interface, receives an input comprising an amino acid sequence for the protein as well as optional pH and temperature values appropriate to an environment of the polypeptide. The output device 108 provides an output comprising one or more of: Aᵢ^{p}, Zᵢ^{p}, Zᵢ^{PS}, Z_{agg}^{S}, and Z_{agg}. For example an aggregation propensity profile or an aggregation propensity graph may be provided (for example as shown in later figures).

Computer system 100 is coupled to a data store 102 which stores hydrophobicity data, β-sheet propensity data (either as propensity data per se or in terms of free energy), optionally α-helix propensity data (see below), and charge data. This data is stored for each amino acid (residue); optionally a plurality of sets of each of these data types is stored corresponding to different values of pH and/or temperature. The computer system, in the illustrated example, is shown interfacing with an α-helix propensity determination system 106 and a local structural; stability determination system 107. One or both of these may be implemented as a separate machine, for example, coupled to computer system 100 over a network, or may comprise a separate or integrated programme running on computer system 100. Whichever method is employed these systems receive sequence data and provides α-helix propensity data and local structural stability data (ln Pᵢ) in return.

As illustrated, computer system 100 may also provide a data output 110, for example Z_{agg}^{S} or Z_{agg}, to an automated peptide synthesiser 112. In this way computer system 100 may be programmed to automatically compare the properties of a number of polypeptides and select one or more of those which are predicted to have favourable properties for automated synthesis. An example of a suitable automated peptide synthesiser is an ABI 433A Peptide Synthesiser (from Applied Biosystems).

α-Helix Propensity

The α-helix propensity may be determined by simply looking up a propensity value for each amino acid of the sequence in a table of propensity values for each of the amino acids. Alternatively an α-helix propensity calculator program may be used, for example the AGADIR code available from http://www.embl-heidelberg.de/Services/serrano/agadir/agadir-start.html, or GOR4 code available from http://npsa-pbil.ibcp.fr/cgi-bin/npsa_automat.pl?page=npsa_gor4.html. Optionally pH and temperature may be taken into account.

β-Sheet Propensity, Hydrophobicity, and Charge

The table below gives scales of hydrophobicity, β-sheet propensity and charge for the 20 natural amino acids.

Scales of hydrophobicity, β-sheet propensity and charge
for the 20 natural amino acids

| amino | | | |
|---|---|---|---|
| acid | hydrophobicity | β-sheet | |
| residue | (kcal mol⁻¹)^{a} | propensity^{b} | charge^{c} |
| Arg (R) | 3.95 | 0.35 | +1 |
| Lys (K) | 2.77 | 0.34 | +1 |
| Asp (D) | 3.81 | 0.72 | −1 |
| Glu (E) | 2.91 | 0.35 | −1 |
| Asn (N) | 1.91 | 0.40 | 0 |
| Gln (Q) | 1.30 | 0.34 | 0 |
| His (H) | 0.64 (2.87)^{d} | 0.37 | 0 (+1)^{d} |
| Ser (S) | 1.24 | 0.30 | 0 |
| Thr (T) | 1.00 | 0.06 | 0 |
| Tyr (Y) | −1.47 | 0.11 | 0 |
| Gly (G) | 0.00 | 0.60 | 0 |
| Pro (P) | −0.99 | n.d. | 0 |
| Cys (C) | −0.25 | 0.25 | 0 |
| Ala (A) | −0.39 | 0.47 | 0 |
| Trp (W) | −2.13 | 0.24 | 0 |
| Met (M) | −0.96 | 0.26 | 0 |
| Phe (F) | −2.27 | 0.13 | 0 |
| Val (V) | −1.30 | 0.13 | 0 |
| Ile (I) | −1.82 | 0.10 | 0 |
| Leu (L) | −1.82 | 0.32 | 0 |

^{a}hydrophobicity values of the 20 amino acid residues at neutral pH based on the partition coefficients from water to octanol. The data are from column 6 of Table 4.8 in ref. 30.
^{b}β-sheet propensities of the 20 amino acid residues normalised from 0 (high β-sheet propensity) to 1 (low β-sheet propensity). The data are from column 4 of Table 1 of ref. 29. The β-sheet propensity of proline is not reported due to the difficulty in determining it experimentally. The β-sheet propensity of glycine is from theoretical calculations.
^{c}values of charge are at neutral pH.
^{d}values in brackets are at a pH lower than 6.0, when the histidine residue is positively charged

With proline, no β-sheet propensity value is available and so a proline residue may be skipped when evaluating equation (1) above, an arbitrary value (eg 1, if β-sheet propensity is expressed in terms of free energy), or one corresponding to another amino acid may be employed.

Pattern Value

A pattern value for each amino acid of the sequence may be determined, for example, by counting the number of polar/non-polar alternations until this reaches 5 or more and then allocating a pattern value (I^{pat}) of say, +1 to each amino acid in the alternating sequence (these values may be normalised so that, say, each amino acid in an alternating sequence of length 5 has a value of +0.2). Alternating hydrophilic (“P”)/hydrophobic (“NP”) patterns give rise to an increased propensity to aggregate. Use of five or more residues is preferred because it appears to be the minimum number of alternating residues that can differentiate between β-sheet promoting (•Δ • Δ •) and α-helix promoting (•Δ• ΔΔ) patterns. Longer alternating sequences may be given larger values, say +2 for a length 9 alternating string of amino acids. Optionally I^{pat} may be given or adjusted by a negative value, say −1, for an aggregation inhibiting pattern (for example a string of hydrophilic amino acids, or a string of some particular amino acids such as prolines).

Residues with hydrophobicity values ≦−0.5 on the Roseman scale [Roseman, M. A., Hydrophilicity of polar amino acid side-chains is markedly reduced by flanking peptide bonds. J Mol Biol, 1988. 200(3): p. 513-22] may be considered hydrophobic and those with values ≧0.5 hydrophilic. Alternatively the following categorisation may be employed: hydrophobic: ala, val, phe, ile, leu, met, tyr, trp; hydrophobic: asp, glu, lys, arg, his, ser, thr, cys, gln, asn; glycine can be hydrophobic or may be considered neutral.

Local Structural Stability (Protection Factor)

The protection factor of a residue i may be defined as the ratio of the intrinsic rate kᵢ^{int} observed in an unstructured peptide, to the observed amide hydrogen exchange rate, kᵢ, Pᵢ=kᵢ^{int}/kᵢ. The local structural stability data (ln Pᵢ) may be determined by determining coefficients of a Fourier transform of the ln P profile from a trained neural network, trained to fit structural data to equilibrium hydrogen exchange measurements:

ln Pᵢ=b_{c} Nᵢ^{c}+bₕ Nᵢ^{h}

where Nᵢ^{c} represents the protection from hydrogen exchange from burial, Nᵢ^{h} is the number of hydrogen bonds for the amide hydrogen of reside i and the parameters b_{c} and bₕ respectively give the free energy contributions of creating a van der Waals contact and a hydrogen bond. Details can be found in CamP; http://www-almost.ch.cam.ac.uk/camp.php.

Results

Experimentally, aggregation-prone regions have been identified by a range of different techniques, including mutational analysis of the kinetics of the aggregation process or of the stability of the amyloid fibrils high-resolution structural analysis of the cores of amyloid fibrils, fluorescence techniques, and the study of the aggregation of peptide fragments extracted from the wild-type proteins. These probes report on different aspects of the dynamics of the aggregation process and of the thermodynamics of the amyloid states. Since the predictions that we perform are based on the analysis of mutational effects on the kinetics of aggregation, we are interested both in assessing the quality of the predictions of the regions that are most important to promote the aggregation process and in exploring the relationship between such aspect and the other factors that may affect the formation and stability of amyloid fibrils.

Prediction of the Aggregation Propensities of Peptides

We first present predictions of the aggregation propensity profiles of four peptides of less than 50 residues that are involved in amyloid disease, Aβ1-42, calcitonin, glucagon, and the second WW domain of CA150 (FIG. 1b). In addition to the intrinsic aggregation-propensity profile, Z^{P}, which is calculated with a procedure described above, we present a second type of profile, Z^{PS}, which takes into account the propensities of the different regions of a polypeptide chain to form stable folded structures (see above).

Aβ¹⁻⁴². We identified two locations of high aggregation propensity (those above the Z^{PS}=1 threshold (upper line), in the central (residues 17-22) and the C-terminal (residues 32-42) regions. Both these regions play an important structural role in the current models of the structures of the Aβ₁₋₄₀ (26) and Aβ1-42 peptides in their amyloid form. The aggregation propensity profile Z^{PS}, which takes into account the tendency of the monomeric form of Aβ₁₋₄₂ to adopt a persistent conformation in solution, reveals that the region of residues 33-38 has a significantly lower propensity for aggregation than that predicted from the intrinsic aggregation propensity profile Z^{P}. This result is in good agreement with the conclusion presented in a recent study in which NM residues 34-37 form a β turn between two short β strands in the monomeric form.

Calcitonin. Human calcitonin is a 32-residue polypeptide hormone involved in calcium regulation and bone dynamics that has been shown to be present as amyloid fibrils in patients with medullar carcinoma of the thyroid. In addition, fibrils can also form in samples prepared in vitro designed for therapeutic use, and represent a considerable limitation on its administration to patients. By calculating the aggregation profile Z^{PS}, we predict a high aggregation potential for the 12-residue N-terminal region and for residues 18-19 and 27-28. Experimentally, K18 and F19 have been identified as key residues in both the bioactivity and self-assembly and the region 15-19 (DFNKF) has been shown to play an active role in oligomerisation and fibril formation in vitro. We did not predict an intrinsic tendency for the monomeric form of this short peptide to form persistent structure, which is consistent with the available experimental evidence. The intrinsic aggregation propensity profile Z^{P} is therefore close to the Z^{PS} profile.

Glucagon. Glucagon is a 29-residue hormone that participates in carbohydrate metabolism and assists in the regulation of the levels of glucose in the blood and thus has been used in the treatment of hypoglycemia. Glucagon has been shown readily to form amyloid fibrils under acidic conditions and the N- and C-terminal regions appear to be important for fibril formation, while the central region (residues 13-18 and 22) plays the major role in determining the morphology of the fibrils themselves. As in the case of Aβ₁₋₄₂ and calcitonin, glucagon is not highly structured in its monomeric form, and consistent with these results the intrinsic aggregation propensity profile Z^{P} is close to the Z^{PS} profile. In agreement with experimental findings, we predict in the N-terminal region (in particular residues T7 and S8) and the C-terminal regions (in particular residue Q24 and W25) to be highly aggregation prone.

CA150.WW2. The second WW domain of human CA150, a protein that is co-deposited with huntingtin in Huntington's disease, is a 40-residue protein that has been shown to form amyloid fibrils in vitro under physiological conditions. The structure of this WW domain in the amyloid protofilament was recently characterised by solid-state NMR spectroscopy, showing that residues 2-14 and 16-29 constitute the core of the fibrils. These experimental results are in agreement with those calculated here, as the regions above the Z^{PS}=1 threshold were identified as those of residues 5-6 and 18-22.

Prediction of the Aggregation Profiles of Globular Proteins

The approach presented here is specifically designed to include the prediction of those regions of the amino acid sequence of a protein that promote its ordered aggregation starting from a globular state. In such cases it is normally necessary to destabilise the structure to enhance the accessibility of the polypeptide main chain and hydrophobic side chains in order for the aggregation process to occur. In this section we discuss two proteins that have been shown to aggregate under such conditions.

Lysozyme. The aggregation propensity profile Z^{PS} calculated by taking into account the structural protection in the native state as predicted from the sequence (lower line in Figure) does not exhibit any region above the Z^{PS}=1 threshold. This result is consistent with the observation that lysozyme must be destabilised in vitro in order to aggregate and that amyloid disease is only found as a result of destabilising familiar mutations. By calculating the intrinsic aggregation propensity profile Z^{P} for wild-type human lysozyme, we identified five aggregation-prone region (residues 42-49, 71-76, 79-85, 92-98 and 109-111) above the Z^{P}=1 threshold. These predictions are of particular interest in the light of recent experimental observations that the region of the sequence that includes residues 32-108 is highly resistant to proteolysis once converted in the amyloid state.

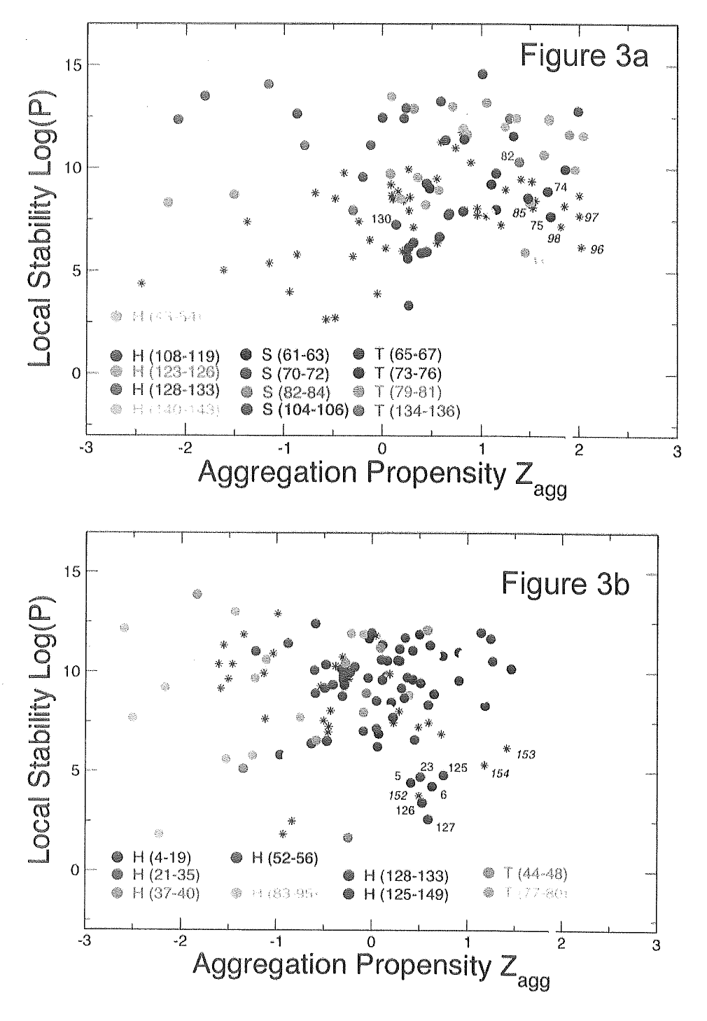

In order to clarify the relationship between the tendencies to remain folded or to aggregate we compared the structural protection and the aggregation propensity at a residue-specific level. The aggregation propensity was measured by the Z^{P} score and the structural protection by the log P score, which provides a prediction of the local stability of the region comprising a particular residue (FIG. 3a). In this type of plot regions of high aggregation propensity and low structural stability in the folded state, which are the most likely to play an important role in first stages of the aggregation process, are found in the right bottom corner of the plot. We predict residues Leu25 (helix) and His78 (turn) to have the highest aggregation propensity and the lowest structural protection. Interestingly, residues Ile56 and Asp67 (strands), which are respectively mutated into Thr56 and His67 in patients suffering from amyloidosis type VIII, show high aggregation propensity and low structural stability.

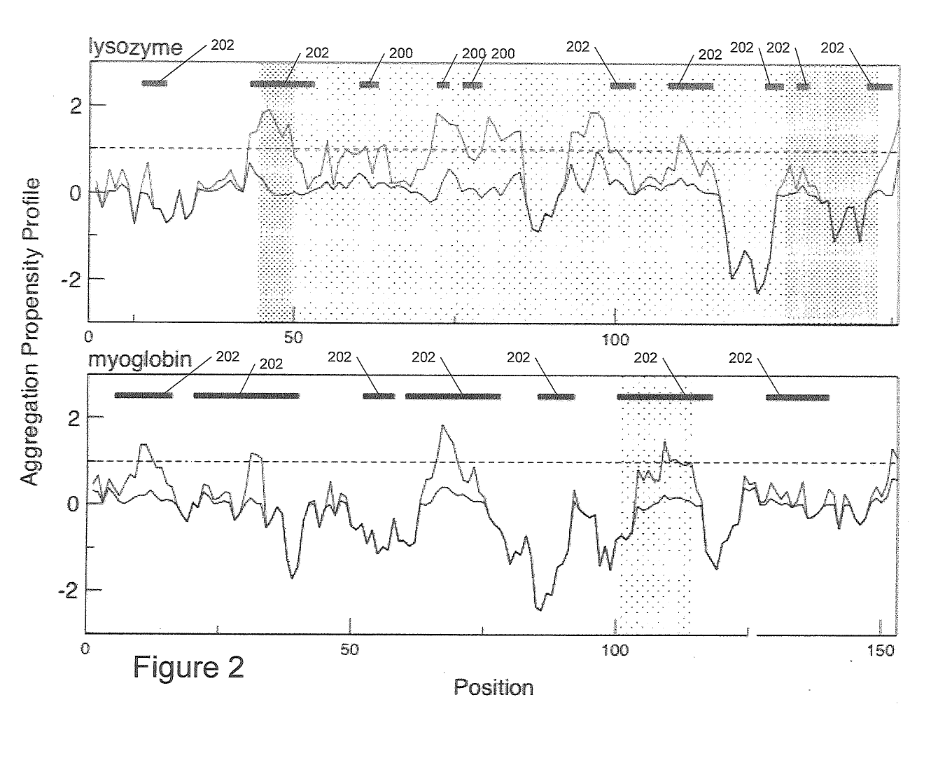

Myoglobin. The aggregation propensity, profile Z^{PS}, calculated by considering the structural protection in the native state, does not exhibit any region above the Z^{PS}=1 threshold, in agreement with the fact that myoglobin should be substantially destabilised in order to aggregate. It is likely that this situation is common for native proteins. As with lysozyme, we identified four regions with a high intrinsic aggregation propensity, i.e. those above the Z^{P}=1 threshold for the upper line in FIG. 2 (residues 9-12, 31-33, 65-70 and 108-114), one of which partially overlaps with a peptide fragment (residues 100-114) found in vitro to be highly prone to aggregation.

In FIG. 3b we compare the aggregation propensity (Z^{P} score) and the structural protection (log P score) at the individual residue level. We predict residues Asp5, Gly6, (helix 4-19), Ala23 (helix 21-35), Gly125, Ala126, and Asp127 (helix 125-149) to have particularly high aggregation propensity and low structural protection.

Prediction of the Aggregation-Prone Regions of Prion Proteins

Human prion protein. A range of human and animal neurodegenerative diseases, the transmissible spongiform encephalopathies (TSEs), is associated with the misfolding and aggregation of mammalian prion proteins. The human prion protein (hPrP) is involved in sporadic, inherited or infectious forms of Creutzfeldt-Jakob disease (CJD), Gerstmann-Straussler-Sheinker disease (GSS) and fatal familial insomnia (FFI). The key event in the pathogenesis associated with these human diseases is the conversion of the normal α-helix-rich and protease-sensitive cellular isoform of the prion protein (hPrP^{C}) into a β-sheet-rich aggregated form (hPrP^{Sc}) that possesses distinct physicochemical properties such as protease resistance, insolubility and potential toxicity. Furthermore, hPrP^{Sc} itself appears to mediate the transmission of TSEs by promoting the conversion of hPrP^{C} into its modified and pathogenic aggregated state.

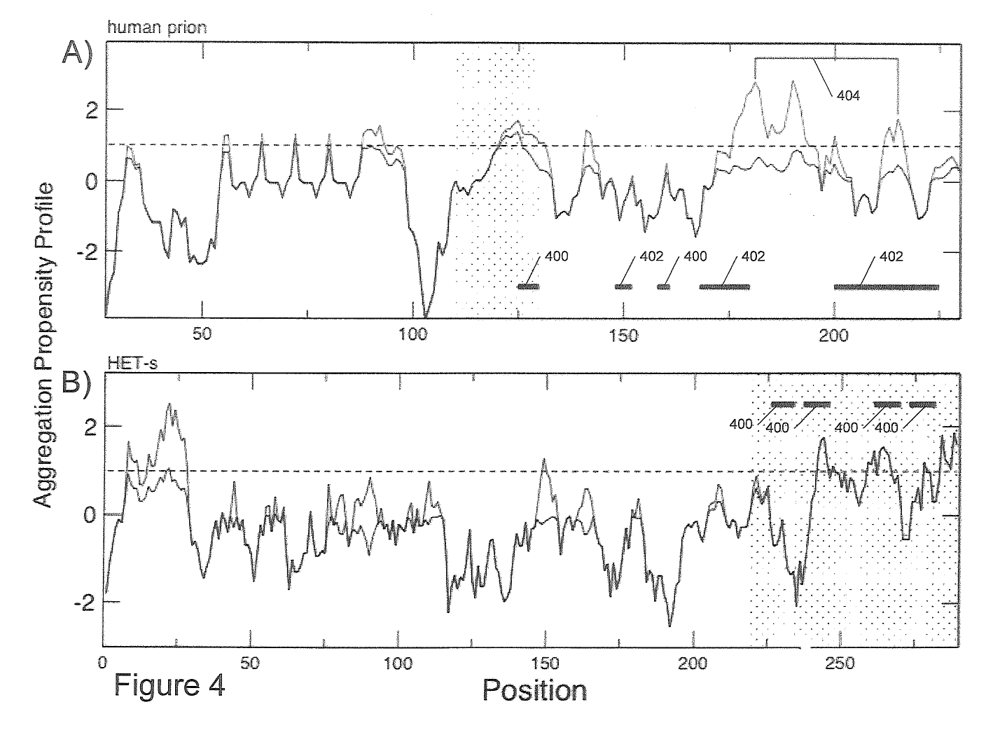

While the mechanism of conversion of hPrP^{C} to hPrP^{Sc} is not known in detail, specific regions of the hPrP^{C} sequence appear to be particularly important in modulating the interaction with hPrP^{Sc} and promoting the process of amyloid formation. In FIG. 3a we show the intrinsic aggregation propensity profile Z^{P} for the sequence of hPrP₍₂₃₋₂₃₁₎. We then took into account the effects of the intrinsic propensities of the various residues to be structured, and hence protected from aggregation (see above). In the latter case, which considers both intrinsic sequence-based propensities and specific structural factors, the region spanning residues 118-128 (dark box in FIG. 4a) corresponds to the highest peak in the entire sequence and the only one to have Z^{PS}>1, suggesting that this region is likely to be the most amyloidogenic segment of the polypeptide chain. The inclusion of terms describing the degree to which the intrinsic propensity for aggregation is modified by the presence of structure is a very important extension of the scope of the prediction method that we described previously for unstructured polypeptides (our previous patent applications, ibid, incorporated by reference). The aggregation profile predicted by considering only the intrinsic physicochemical factors (FIG. 4a) identifies the region 180-186, corresponding to α-helix II, as the most prominent amyloidogenic region. However, this region is highly structured in the hPrPC form and does not appear from experimental data to be as important for aggregation as the region of residues 113-127. The similarity in the Z^{P} and the Z^{PS} profiles for residues 1-125 is in agreement with the experimental observation that this region is not structured. In addition, the presence of the disulfide bond C179-C214 appears to play an important role in stabilizing this highly aggregation-prone region and inhibits the formation of intermolecular interactions. We also calculated a significant propensity for aggregation in the vicinity of the copper-binding region comprising the four tandem repeats of the octapeptide sequence PHGGGWGQ, in agreement with the observation that this region may play an important role in the oligomerisation process of this protein.

The predicted aggregation propensity profiles Z^{P} and Z^{PS} correlate well with experimental data on the in vitro aggregation behaviour of hPrP fragments. Peptides hPrP₁₀₆₋₁₁₄, hPrP₁₀₆₋₁₂₆, hPrP₁₁₃₋₁₂₆ and hPrP₁₂₇₋₁₄₇ of recombinant hPrP all have high propensities to form amyloid fibrils. hPrP₁₀₆₋₁₂₆ has a particularly high intrinsic ability to polymerize into straight and unbranched fibrils and induces apoptosis in primary rat hippocampal cultures (25). hPrP₁₁₃₋₁₂₆ is also able to aggregate readily although the fibrils in these preparations are less abundant at equal initial peptide concentrations and are reduced both in length and diameter relative to hPrP₁₀₆₋₁₂₆. hPrP₁₀₆₋₁₁₄ and hPrP₁₂₇₋₁₄₇ have a lower tendency to aggregate than hPrP₁₀₆₋₁₂₆, although the former converts into fibrils that are morphologically similar to those formed by hPrP₁₀₆₋₁₂₆ whilst the latter forms twisted fibrillary structures. A recent report has identified two other peptide fragments, hPrP₁₁₉₋₁₂₆ and hPrP₁₂₁₋₁₂₇, which can readily form amyloid-like fibrils and can be cytotoxic to astrocytes. These fragments include, at least in part, the region 118-128 of the sequence (FIG. 4a).

The calculations described here for the human prion protein support the view that structural factors are important in determining the aggregation rates of proteins that self assemble via aggregation-prone partially folded states. We found that all the mutations occurring in CJD (http://www.expasy.org/uniprot/PRIO_HUMAN), with exception of D178N and V180I, have higher aggregation propensity Z_{agg}^{S} (Equation 7) than the wild type (Table 1).

TABLE 1

| | V180I | D178N | WT | V203I | E200K | R208K | M129V | V210I | E196K | E211Q |
|---|---|---|---|---|---|---|---|---|---|---|
| Z_{agg} | 0.96 | 0.96 | 0.97 | 0.96 | 0.95 | 0.97 | 0.98 | 0.97 | 0.94 | 1.00 |
| Z^{s}_{agg} | 0.46 | 0.50 | 0.51 | 0.52 | 0.54 | 0.54 | 0.60 | 0.61 | 0.62 | 0.66 |

Overall aggregation propensity, Z^{s}_{agg}, for mutations associated to the Creutzfeldt-Jakob disease (http://www.expaasy.org/uniprot/PRIO-HUMAN). All the mutations except D178N and V180I have higher aggregation propensity than the wild type.

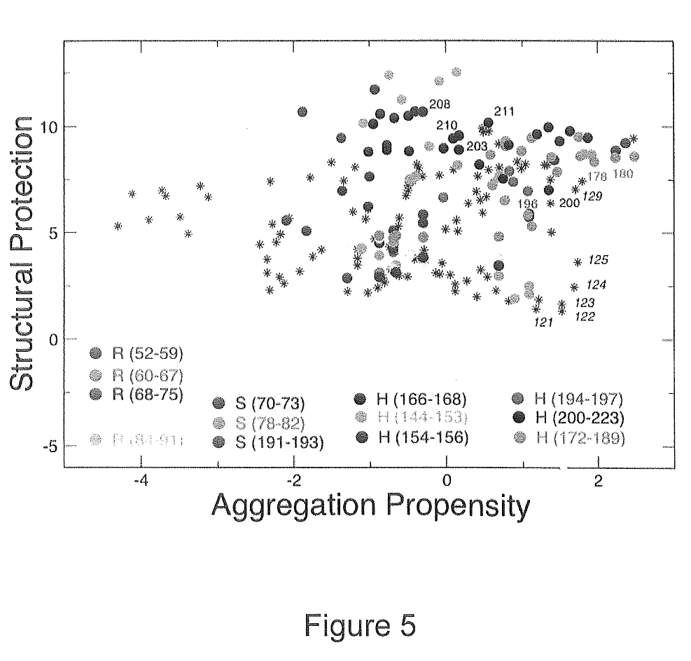

We predict mutations D178N and V180I to increase the protection of helix 172-189, resulting in a decrease of the overall aggregation propensity of the protein. The comparison of the aggregation propensity (Z^{P} score) and the structural stability (log P score) at the individual residue level is shown in FIG. 5. We observe that the region of residues 120-123 has the highest aggregation propensity and the lowest structural protection, followed by the region of the repeat 84-91. We also label the positions associated to the CJD mutations reported in Table 1 above.

HET-s. HET-s of the yeast Podospora anserine is a prion protein involved in heterokaryon incompatibility and which is not associated with disease. HET-s has been shown to form amyloid fibrils, whose structures have been characterised through solid-state NMR, in conjunction with site-directed fluorescence labeling and a hydrogen exchange protocol. In the resulting structural model of the fibrils from the C-terminal fragment of HET-s (residues 218-289) each molecule contributes four β-strands, with strands 1 and 3 (residues 226-234 and 262-270) forming a parallel β-sheet and strand 2 and 4 (residues 237-245 and 273-282) forming another parallel β-sheet located about 10 Å away. These β-strands are connected by two short loops between β1and β2, and β3 and β4 respectively, and by an unstructured 15-residue segment between β2 and β3.

Calculation of the intrinsic aggregation propensity profile Z^{P} (FIG. 4b) reveals a high aggregation propensity in the regions of residues 5-22 and 245-289. The monomeric form of HET-s appears to be structured in the region of residues 1-227 and rather unstructured in the region of residues 228-289 (9). Consistently with these results, we determined a much lower propensity for aggregation in the C-terminal region through the Z^{PS} profile (FIG. 4b), which results in part from the very high structural protection predicted for this region by the CamP method (ibid). The region encompassing residues 228-289 is therefore predicted to be the principal aggregation-prone region. This fragment, in contrast with fragment 1-227, retains the ability to form fibrils in vitro, catalyses efficiently the aggregation of full-length HET-s and is able to induce prion propagation in vivo. In addition, limited proteolysis experiments indicate that the region of residues 218-289 is in the fibril core. Three of the four β-strands identified experimentally (residues 226-234, 237-244, 262-271 and 273-282) as those forming the core of the cross-β structure correspond to the major three peaks (residues 242-245, 260-267 and 278-289) in the aggregation propensity profile Z^{PS} of HET-s (FIG. 4b). We therefore suggest that β-strand 1 plays an important thermodynamic role in stabilising the structure of the amyloid fibril, it is not likely to be directly involved in the process of aggregation.

We have described in this paper a method for predicting the regions of the sequences of both structured and partially structured proteins that are most important in promoting their aggregation. Our analysis reveals that the regions that promote aggregation, even from globular states, can be identified on the basis of the knowledge of the amino acid sequence. The methodology that we have presented is general and is based on the idea that the sequence of a protein determines its behaviour in the case of both folding and misfolding. The possibility provided by methods such as the one that we have presented here to predict the aggregation-promoting regions for natively unfolded polypeptide chains, for globular proteins and for systems that contain both folded and unfolded domains may be of significant value in developing rational approaches to the avoidance of aggregation in biotechnology and to the treatment of aggregation diseases as it identifies the major factors determining aggregation as well as the regions in which these factors are prevalent.

No doubt many other effective alternatives will occur to the skilled person. It will be understood that the invention is not limited to the described embodiments and encompasses modifications apparent to those skilled in the art lying within the spirit and scope of the claims appended hereto.

1-22. (canceled)

23. A method of identifying one or more regions in the amino acid sequence of a protein which, in the folded protein, are predicted to promote aggregation, the method comprising:
determining, for amino acid positions (i) along said sequence, a local propensity for aggregation (Aᵢ) at a said amino acid position, said local propensity for aggregation being determined by a combination of a hydrophobicity value, an α-helix propensity value, a β-sheet propensity value, a charge value and a pattern value for said amino acid position;
determining local structural stability values for said amino acid positions, a said local structural stability value comprising a measure of local structural stability at a said amino position; and
combining said determined local propensities for aggregation at said amino acid positions and said local structural stability values at said amino acid positions to identify one or more regions in said amino acid sequence which, in said folded protein, are predicted to promote aggregation.

24. A method as claimed in claim 23 wherein said combining comprises modifying said determined local propensities for aggregation at said amino acid positions using said local structural stability values at said amino acid positions to determine modified local propensities for aggregation defining an aggregation propensity profile for said folded protein, said aggregation propensity profile comprising data defining variations in said modified local propensities for aggregation with amino acid positions along said sequence; the method further comprising identifying said one or more regions in said amino acid sequence which, in said folded protein, are predicted to promote aggregation from said aggregation propensity profile.

25. A method as claimed in claim 24 further comprising selecting, for said identifying, only regions of said aggregation propensity profile having greater than a threshold local propensity for aggregation.

26. A method as claimed in claim 24 wherein said modifying of said determined local propensities for aggregation at said amino acid positions comprises modulating said determined local propensities for aggregation at said amino acid positions by logarithm Pᵢ where Pᵢ comprises a structural protection factor for the amino acid at position i in said sequence.

27. A method as claimed in claim 23 wherein said measure of local structural stability at a said amino position comprises a measure of propensity of said folded protein at a said amino acid position to remain in a folded state.

28. A method as claimed in claim 23 wherein each said local structural stability value at a said amino acid position is determined from said amino acid sequence of said protein.

29. A method as claimed in claim 23 wherein a said local structural stability value at a said amino acid position includes a charge gatekeeping value dependent on a total local charge within a window to either side of said amino acid position.

30. A method as claimed in claim 23 comprising:
determining, for a plurality of positions, i, along said sequence, a value of pᵢ^{agg}, where pᵢ^{agg} represents an intrinsic aggregation propensity of an amino acid at position i and comprises a function of pₕ, pₛ, p_{hyd} and p_{c} and pₕ, pₛ, p_{hyd} and p_{c} are, respectively, an α-helix propensity value, a β-sheet propensity value, a hydrophobicity value, and a charge value for an amino acid at a said position i along said sequence;
determining, for a plurality of positions, i, along said sequence, a value of Aᵢ^{p}, where Aᵢ^{p} is determined from
$${α}_{1} {\sum }_{window 1} {p}_{i}^{agg} + {α}_{pat} {I}_{i}^{pat} + {α}_{gk} {I}_{i}^{gk}$$
where
$${\sum }_{window 1}$$
denotes a first sum over amino acid positions in a first window to either side of position i, Iᵢ^{pat} is a pattern value representing a pattern of one or both of hydrophilic and hydrophobic amino acids at position i, Iᵢ^{gk} is a charge value representing a charge flanking or inside a said pattern, and wherein αᵢ, αₚₐₜ and α_{gk} are scaling factors; and
determining an aggregation propensity profile for said protein from values of Aᵢ^{p} for said plurality of positions i along said sequence, said aggregation propensity profile comprising data identifying a variation of relative aggregation propensity with position along said sequence.

31. A method as claimed in claim 30 wherein said determining of said charge value Iᵢ^{gk} comprises determining a value for
$${\sum }_{window 2} charge$$
where
$${\sum }_{window 2} charge$$
denotes a second sum over amino acid positions in a second window to either side of position i, said sum comprising a sum of charges at said amino acid positions in said second window.

32. A method as claimed in claim 30 wherein said determining of said aggregation propensity profile comprises determining from each value of Aᵢ^{p} a value of Zᵢ^{PS} for said positions i where Zᵢ^{PS} is determined by multiplying a value dependent on Aᵢ by
$$( {α}_{2} - \frac{logarithm {P}_{i}}{{α}_{3}} )$$
where α₂ and α₃ are scaling factors and Pᵢ comprises a structural protection factor for position i, said structural protection factor being dependent on a degree to which a structure of said protein at position i is protected, in its folded state, from aggregation.

33. A method as claimed in claim 32 wherein said value dependent on Aᵢ comprises a value for Zᵢ^{P} for said positions i, where Zᵢ^{P} represents a normalized intrinsic aggregation propensity for position i.

34. A method as claimed in claim 23 for determining the aggregation propensity of a protein, the method comprising using the method of claim 23 to identify one or more regions in the amino acid sequence of a protein which, in the folded protein, are predicted to promote aggregation, and then summing either aggregation propensity data determined from said local propensity for aggregation or values of Aᵢ, wherein said summing comprises summing over substantially only said identified regions.

35. A method as claimed in claim 34 for determining the aggregation propensity of a protein, the method further comprising controlling automatic polypeptide synthesis apparatus to determine said aggregation propensity of said protein, using said determined aggregation propensity to select a polypeptide for synthesis, and then controlling said automatic polypeptide synthesis apparatus to make said selected polypeptide.

36. A method as claimed in claim 23 for making a protein having an amino acid sequence, the method being characterised by using the method of claim 23 to identify either said one or more regions in the amino acid sequence of a protein which, in the folded protein, are predicted to promote aggregation or an overall said aggregation propensity of said protein.

37. A method as claimed in claim 23 for determining the toxicity data for a protein, the method comprising using the method of claim 23 to identify either said one or more regions in the amino acid sequence of a protein which, in the folded protein, are predicted to promote aggregation or an overall said aggregation propensity of said protein, and then using said identified regions or said overall said aggregation propensity of said protein to determine said toxicity data.

38. A method as claimed in claim 23 for identifying a drug target in a protein, said drug target comprising a target portion of an amino acid sequence of said protein, the method comprising using the method of claim 23 to identify said one or more regions in the amino acid sequence of a protein which, in the folded protein, are predicted to promote aggregation, and then using said identified regions to identify a said target portion of said amino acid sequence for targeting by a drug.

39. A method as claimed in claim 38 for identifying a drug which interacts with a protein, the method comprising using the method of claim 38 to identify a drug target in said protein, and then identifying a drug which interacts with said target portion of said amino acid sequence.

40. A method as claimed in claim 39 wherein said identifying comprises screening candidate drugs against said drug target.

41. A method as claimed in claim 23, wherein the method is computerised, the method further comprising outputting the results of at least one of the steps to at least one of a display and a memory.

42. A carrier carrying computer program code for identifying one or more regions in the amino acid sequence of a protein which, in the folded protein, are predicted to promote aggregation, the code comprising code to:
determine, for amino acid positions (i) along said sequence, a local propensity for aggregation (Aᵢ) at a said amino acid position, said local propensity for aggregation being determined by a combination of a hydrophobicity value, an α-helix propensity value, a β-sheet propensity value, a charge value and a pattern value for said amino acid position;
determine local structural stability values for said amino acid positions, a said local structural stability value comprising a measure of local structural stability at a said amino position; and
combine said determined local propensities for aggregation at said amino acid positions and said local structural stability values at said amino acid positions to identify one or more regions in said amino acid sequence which, in said folded protein, are predicted to promote aggregation.

43. A carrier carrying computer program code as claimed in claim 42 incorporated into automatic laboratory equipment wherein said equipment is configured for control by said computer program code to:
determine, for amino acid positions (i) along said sequence, a local propensity for aggregation (Aᵢ) at a said amino acid position, said local propensity for aggregation being determined by a combination of a hydrophobicity value, an α-helix propensity value, a β-sheet propensity value, a charge value and a pattern value for said amino acid position;
determine local structural stability values for said amino acid positions, a said local structural stability value comprising a measure of local structural stability at a said amino position; and
combine said determined local propensities for aggregation at said amino acid positions and said local structural stability values at said amino acid positions to identify one or more regions in said amino acid sequence which, in said folded protein, are predicted to promote aggregation.

44. A method of identifying one or more regions in the amino acid sequence of a protein which, in the folded protein, are predicted to promote aggregation, the method comprising:
determining, for a plurality of positions, i, along said sequence, a value of pᵢ^{agg}, where pᵢ^{agg} represents an intrinsic aggregation propensity of an amino acid at position i and comprises a function of pₕ, pₛ, p_{hyd} and p_{c} and pₕ, pₛ, p_{hyd} and p_{c} are, respectively, an α-helix propensity value, a β-sheet propensity value, a hydrophobicity value, and a charge value for an amino acid at a said position i along said sequence;
determining, for a plurality of positions, i, along said sequence, a value of Aᵢ^{p}, where Aᵢ^{p} is determined from
$${α}_{1} {\sum }_{window 1} {p}_{i}^{agg} + {α}_{pat} {I}_{i}^{pat} + {α}_{gk} {I}_{i}^{gk}$$
where
$${\sum }_{window 1}$$
denotes a first sum over amino acid positions in a first window to either side of position i, Iᵢ^{pat} is a pattern value representing a pattern of one or both of hydrophilic and hydrophobic amino acids at position i, Iᵢ^{gk} is a charge value representing a charge flanking or inside a said pattern, and wherein α₁, αₚₐₜ and α_{gk} are scaling factors; and
determining an aggregation propensity profile for said protein from values of Aᵢ^{p} for said plurality of positions i along said sequence, said aggregation propensity profile comprising data identifying a variation of relative aggregation propensity with position along said sequence.

45. A method as claimed in claim 44 wherein said determining of said charge value Iᵢ^{gk} comprises determining a value for
$${\sum }_{window 2} charge$$
where
$${\sum }_{window 2} charge$$
denotes a second sum over amino acid positions in a second window to either side of position i, said sum comprising a sum of charges at said amino acid positions in said second window.

46. A method as claimed in claim 44 wherein said determining of said aggregation propensity profile comprises determining from each value of Aᵢ^{p} a value of Zᵢ^{PS} for said positions i where Zᵢ^{PS} is determined by multiplying a value dependent on Aᵢ by
$$( {α}_{2} - \frac{logarithm {P}_{i}}{{α}_{3}} )$$
where α₂ and α₃ are scaling factors and Pᵢ comprises a structural protection factor for position i, said structural protection factor being dependent on a degree to which a structure of said protein at position i is protected, in its folded state, from aggregation.

47. A method as claimed in claim 46 wherein said value dependent on Aᵢ comprises a value for Zᵢ^{P} for said positions i, where Zᵢ^{P} represents a normalized intrinsic aggregation propensity for position i.

48. A method as claimed in claim 44 for determining the aggregation propensity of a protein, the method comprising using the method of claim 44 to identify one or more regions in the amino acid sequence of a protein which, in the folded protein, are predicted to promote aggregation, and then summing either aggregation propensity data determined from said local propensity for aggregation or values of Aᵢ, wherein said summing comprises summing over substantially only said identified regions.

49. A method as claimed in claim 44 for making a protein having an amino acid sequence, the method being characterised by using the method of claim 44 to identify either said one or more regions in the amino acid sequence of a protein which, in the folded protein, are predicted to promote aggregation or an overall said aggregation propensity of said protein.

50. A method as claimed in claim 44 for determining the toxicity data for a protein, the method comprising using the method of claim 44 to identify either said one or more regions in the amino acid sequence of a protein which, in the folded protein, are predicted to promote aggregation or an overall said aggregation propensity of said protein, and then using said identified regions or said overall said aggregation propensity of said protein to determine said toxicity data.

51. A method as claimed in claim 44 for identifying a drug target in a protein, said drug target comprising a target portion of an amino acid sequence of said protein, the method comprising using the method of claim 44 to identify said one or more regions in the amino acid sequence of a protein which, in the folded protein, are predicted to promote aggregation, and then using said identified regions to identify a said target portion of said amino acid sequence for targeting by a drug.

52. A method as claimed in claim 51 for identifying a drug which interacts with a protein, the method comprising using the method of claim 51 to identify a drug target in said protein, and then identifying a drug which interacts with said target portion of said amino acid sequence.

53. A method as claimed in claim 52 wherein said identifying comprises screening candidate drugs against said drug target.

54. A method as claimed in claim 44, wherein the method is computerised, the method further comprising outputting the results of at least one of the steps to at least one of a display and a memory.

55. A method of determining the overall aggregation propensity of a folded protein, the method comprising:
identifying one or more regions in the amino acid sequence of a protein which, in the folded protein, are predicted to promote aggregation taking into account one or both of a local hydrogen exchange and the suppression of an aggregation-inducing amino acid pattern by local charge; and then
summing aggregation propensity data determined from values of a local propensity for aggregation (Aᵢ) at a plurality of amino acid positions (i) along said sequence;
wherein said summing comprises summing over substantially only said identified regions.

56. A method as claimed in claim 55, wherein the method is computerised, the method further comprising outputting the results of at least one of the steps to at least one of a display and a memory.
